(19) United States
(12) Patent Application Publication
(10) Pub. No.: US 2026/0224771 A1
(43) Pub. Date: Aug. 6, 2026

(54) DEVICE FOR DIFFUSING VOLATILE SUBSTANCES

(71) Applicant: ZOBELE HOLDING SPA, Trento (IT)

(72) Inventors: José María García Jiménez, Barcelona (ES); Roberto Camarero Díez, Barcelona (ES); Alberto José Sancho Marzo, Barcelona (ES)

(73) Assignee: ZOBELE HOLDING SPA, Trento (IT)

(21) Appl. No.: 19/147,633

(22) PCT Filed: Jan. 15, 2024
(86) PCT No.: PCT/EP2024/050766

§ 371 Date: Jul. 11, 2025

(30) Foreign Application Priority Data
Jan. 13, 2023 (ES) P202330019

(51) Int. Cl. A61L 9/14; B65D 83/26

(52) U.S. Cl. CPC A61L 9/14; B65D 83/262; A61L 2209/111; A61L 2209/134; A61L 2209/20

(57) ABSTRACT

The device for diffusing volatile substances comprises a container containing the volatile substances to be diffused, a valve in the container through which the volatile substances are diffused, an actuating system which actuates the valve, and a control system in communication with the actuating system which determines the actuating time of the valve for each diffusion of the volatile substances, the actuating time of the valve being variable, and a control system in communication with the actuation system, which determines the actuation time of the valve for each diffusion of the volatile substances, the actuation time of the valve for each diffusion of the volatile substances being variable. It provides a device for diffusing volatile substances that allows the use of a refill with a naturally occurring propellant composition such as nitrogen, carbon dioxide, nitrous oxide or compressed air without impairing spray consistency.

### OBJECT OF THE INVENTION

The present invention refers to a device for diffusing volatile substances, which allows the use of a refill with a propellant composition of natural origin, such as nitrogen, carbon dioxide or compressed air.

### BACKGROUND TO THE INVENTION

There are many types of products that release volatile substances into the environment, so as to have a continuous action over several days, weeks or months. Among these products, automatic aerosol dispensers stand out. These devices regularly release doses of volatile substances such as perfumes, insecticides, or other types of active ingredients. These devices consist of an aerosol can with a shut-off valve and an activation mechanism of this valve that is regularly activated.

These automatic aerosol dispensers have several advantages over the more classical diffusers based on heating systems and refills with liquid and wick:
On the one hand, they require a much lower level of energy, which makes it possible to have portable devices with batteries, and therefore do not need to be connected to the domestic electricity grid.
Moreover, the doses released are very consistent, both in chemical composition and quantity, allowing for consistent environmental performance throughout the life of the refill.

In the existing solutions, the correct functioning is related to the use of hydrocarbon-based propellants, which allow the maintenance of optimum physical properties for adequate performance.

In current spraying cases, the pulses are factory preset and constant, ensuring an acceptable distribution of volatile substance.

However, in these types of devices currently known, the use of refills with liquid hydrocarbon propellant, which is flammable and harmful to the environment due to its emissions, is necessary to maintain a constant pressure inside the refill. An increasing demand for more environmentally friendly products is being observed and aerosol container solutions based on naturally occurring propellant are being seen.

However, these propellants are not as efficient as hydrocarbon-based propellants and the spray output with each activation of the valve decreases over the life of the refill. When using these aerosols manually, the user may notice this drop in atomization and act accordingly to correct this effect. However, in the case of an automatic dispenser, this drop in internal pressure will result in a drop in atomized dose over the life of the product and therefore a drop in performance,

### DESCRIPTION OF THE INVENTION

Therefore, an objective of the present invention is to provide a device for diffusing volatile substances that allows the use of a refill with a propellant composition of natural origin, such as nitrogen, carbon dioxide, nitrous oxide or compressed air without impairing the consistency of the spray.

With the device for diffusing volatile substances of the invention, the aforementioned disadvantages are solved, presenting other advantages that will be described below.

The device for diffusing volatile substances according to the present invention is described in claim 1, and the dependent claims include additional features which are optional.

In particular, the device for diffusing volatile substances according to the present invention comprises:
a container that holds the volatile substances to be diffused;
a valve in the container, through which the volatile substances are diffused;
a mechanical actuating system, which actuates the valve,
wherein the device for diffusing volatile substances also comprises a control system in communication with the actuation system, which determines the valve actuation time for each diffusion of the volatile substances, wherein the valve actuation time for each diffusion of the volatile substances can be variable.

Furthermore, advantageously, the device for diffusing volatile substances according to the present invention may comprise a detector, which communicates such information to the control system to determine the valve actuation time at each diffusion of the volatile substances.

This detector can perform different functions, such as detecting the presence of the container, the type of container or the amount of volatile substances present inside the container.

Preferably, the detector detects the type of container by means of a container identification, such as a barcode, QR, RFID tag, or the like.

Preferably, the detector detects the amount of volatile substances present inside the container by detecting the internal pressure of the container, the weight of the container contents, and/or electronic characteristics of container contents, such as impedance, conductivity, resistance and/or intensity.

According to a preferred embodiment, the actuation system comprises a plurality of gears and an actuator, the actuator acting directly on the valve.

Advantageously, the container houses a pressurized gas of natural origin, such as nitrogen, carbon dioxide or compressed air.

The device for diffusing volatile substances allows the use of a refill with a propellant composition wholly or partly of natural origin, such as nitrogen, carbon dioxide or compressed air, in its gaseous phase.

In addition to the environmental advantages of not using liquefied gases of petrochemical origin, other advantages are obtained from the point of view of user safety, as these gases have no flammable properties.

With the device for diffusing volatile substances according to the present invention, the pressure drop effect is compensated by using a gas of natural origin, reactivating the contents of the refill through a longer spray duration, and dispensing with a metered dosing valve.

Consequently, with the device according to the present invention, the use of aerosols filled with propellant gases of natural origin is allowed, with the consequent benefit for the environment, without impacting on the quality of the user experience, as they remain at similar levels to current diffuser devices using liquefied gases of petrochemical origin.

### BRIEF DESCRIPTION OF THE DRAWINGS

For a better understanding of what has been explained above, some drawings are included in which, schematically and only by way of a non-limiting example, a practical case of embodiment is represented.

FIG. 1 is a schematic perspective view of the device for diffusing volatile substances according to the present invention.

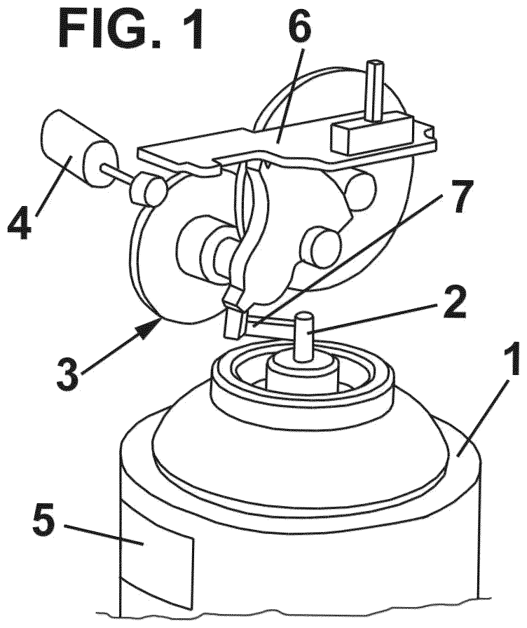

### DESCRIPTION OF A PREFERRED EMBODIMENT

The device for diffusing volatile substances according to the present invention is an electrical device for diffusing volatile substances by automatic activation of an aerosol can, powered, for example, by batteries.

The device according to the present invention allows to optimize the dosage of volatile substances by compensating the pressure loss to diffuse the desired amount of volatile substance into the environment.

According to the embodiment depicted, the device according to the present invention comprises a refill or container (1), which houses the volatile substances to be diffused and a pressurized gas, and a valve (2), which valve (2) may have a flow reduction feature.

Unlike currently known device for diffusing volatile substances, the pressurized gas inside the container (1) is a gas of natural origin, such as nitrogen, carbon dioxide or compressed air.

It also comprises an actuation system (3), activated by a motor (4), e.g. a DC motor, to mechanically open and close the valve (2) integrated in the container (1). If desired, the actuation system (3) could comprise a locking or travel stop element to improve its precision and enable rapid actuation.

The actuation system (3) preferably comprises a plurality of gears and an actuator (7), the actuator (7) acting directly on the valve (2).

The device according to the present invention also comprises a detector (5) of the remaining quantity of volatile substances in the container (1) or of the type of container (1) being used, and a control system (6), for example of an electronic type, which applies a dosing cycle according to the information received from the detector (5), and a nozzle, not shown in the drawings, for the diffusion of the volatile substances once they have passed the valve (2).

The control system (6) performs the programmed activation of the diffusion of volatile substances based on the data received on the remaining quantity of volatile substances in the container (1) or the type of container (1) being used, with the aim of dispensing the desired quantity of volatile substances during a spray cycle, i.e. a given period of time, which compensates for the loss of pressure of the pressurized gas contained in the container (1), due to the expansion of the same inside the container (1).

The detector (5) of the amount of volatile substances remaining in the container (1) may be of any suitable type, such as, for example, a detector of the internal pressure of the container (1), a detector of the weight of the contents of the container (1), or a detector of the electronic characteristics, such as impedance, conductivity, resistance, current, of the contents remaining in the container (1), or any combination thereof.

Advantageously, with the device for diffusing volatile substances according to the present invention, by being able to precisely change the diffusion of volatile substances according to the quantity of volatile substances present inside the container and their possible pressure drop, it facilitates the dosage of the volatile substance in a pressurized container by means of natural compressed air, without the need to include a dosage type valve measured in the container, with consequent cost savings.

According to a first example, the volatile substance dosing device according to the present invention may comprise a detector (5) of the type of container (1) being used, which container (1) can be identified by means of a barcode, QR, RFID tag, NFC or microchip. Upon identification of the container (1), a pre-installed program is activated, which was made considering the pressure loss for the volatile substances contained in that container (1). This program will vary the activation time of the device through the control system (6), in order to always dispense the same amount of volatile substances, increasing the dosing time.

Alternatively, this pressure drop compensation can be carried out by means of a pre-installed, universal program without the need for identification of the container (1).

According to a second example, the detector (5) detects certain parameters of the container (1) to determine, in a real way, the quantity of volatile substances present inside the container (1). For this determination, the detector (5) can detect the weight, conductivity, impedance, conductivity resistance, etc., sending the reading to be processed by the control system (6), and determine the dispensing activation times accordingly, to compensate for this pressure drop, and always dispense the same amount of volatile substances throughout the life of the container (1) or refill.

Although reference has been made to a specific embodiment of the invention, it is obvious to a person skilled in the art that the device for the evaporation of volatile substances described is susceptible to numerous variations and modifications, and that all the details mentioned can be replaced by technically equivalent ones, without departing from the scope of protection defined by the appended claims.

1. Device for diffusing volatile substances, comprising:
a container containing the volatile substances to be diffused;
a valve in the container, through which the volatile substances are diffused;
an actuation system which actuates said valve,
characterized in that the device for diffusing volatile substances also comprises a control system in communication with the actuation system, which determines the actuation time of the valve for each diffusion of the volatile substances, whereby this valve actuation time can be variable for each diffusion of the volatile substances.

2. Device for diffusing volatile substances according to claim 1, comprising a detector, which detects the presence of the container, the type of container or the quantity of volatile substances present inside the container, which detector communicates such information to the control system to determine the valve actuation time for each diffusion of the volatile substances.

3. Device for diffusing volatile substances according to claim 2, wherein the detector detects the type of container by means of an identification of the container.

4. Device for diffusing volatile substances according to claim 2, wherein the detector detects the amount of volatile substances present inside the container by detecting the internal pressure of the container, the weight of the container contents, and/or electronic characteristics of container contents (1).

5. Device for diffusing volatile substances according to claim wherein the actuation system comprises a plurality of gears and an actuator, the actuator acting directly on the valve.

6. Device for diffusing volatile substances according to claim 1, wherein the container contains a pressurized gas of natural origin.

7. Device for diffusing volatile substances according to claim 6, wherein the pressurized gas is nitrogen, carbon dioxide or compressed air.
